(19) United States
(12) Patent Application Publication
(10) Pub. No.: US 2019/0159731 A1
(43) Pub. Date: May 30, 2019

(54) Tattoo biosensor and health monitoring system

(71) Applicant: Umana Medical Technologies Ltd., Mriehel (MT)

(72) Inventors: Paolo Cavallari, Milano (IT); Adrian Attard Trevisan, Rabat (MT); Krystle Attard Trevisan, Rabat (MT); Francesco Greco, Graz (AT); Laura Ferrari, Livorno (IT); Vincenzo Varriale, Rome (IT)

(21) Appl. No.: 15/827,014

(22) Filed: Nov. 30, 2017

(51) Int. Cl. A61B 5/00; A61B 5/0408; A61B 5/0492; A61B 5/0478

(52) U.S. Cl. CPC A61B 5/68335; A61B 5/0006; A61B 5/04085; A61B 5/0452; A61B 5/0478; A61B 5/747; A61B 5/746; A61B 5/0492

(57) ABSTRACT

A conformal tattoo biosensor device includes a pattern of sensor regions formed of a conductive polymer. In embodiments, the conductive polymer may have up to six sensor regions. The pattern is electrically connected to a contact region which is electrically connectable to a wearable signal monitor. The monitor is suitable for transmitting ECG, EEG, or EMG signals. In a monitoring system, the monitor wirelessly transmits signals to a mobile communication device for processing. The mobile communication device transmits signals to a monitor network which may include medical personnel and caregivers. A network module may allow automatic medical alerts, monitoring, and further signal processing.

### CROSS REFERENCE TO RELATED APPLICATION

NONE

### TECHNICAL FIELD

The present invention pertains generally to electrophysiological monitoring, and more particularly to temporary tattoo biosensors and health monitoring systems therewith.

### BACKGROUND OF THE INVENTION

Conventional electrophysiological monitoring methods, such as electrocardiography (ECG or EKG), electromyography (EMG), and electroencephalography (EEG), use conductive electrodes adhered to the skin of a patient with an adhesive or electrolytic gel. The electrodes are wired to a data acquisition unit. The size and wired connections of such systems make them impractical and inconvenient for long-term or mobile use. In addition, gels have a short useful lifetime before drying out. Furthermore, adhesives, gels, and the preparation method used to attach gelled sensors often irritate the patient's skin.

Recently, conductive polymer films have been demonstrated to have properties suitable for use as biosensors (Greco et al., 2011). Conformable tattoo biosensors having submicrometric thickness were demonstrated in polymer films (Zucca et al., 2015). Other tattoo biosensors have been demonstrated using silver nanoparticles (Casson et al., 2016), polymer-enhanced carbon (Bareket et al., 2016), and graphene (Ameri, et al., 2017). Demonstrated applications include ECG, EMG, and EEG monitoring.

### BRIEF SUMMARY OF THE INVENTION

Generally speaking, the present disclosure teaches a conformable temporary tattoo biosensor having a submicrometric thickness. The sensor device is readily transferred from a substrate sheet to the skin of a patient, and is highly conformable, enabling better impedance response than conventional adhesive or pre-gelled sensors. The sensor regions (or electrode surfaces) of the tattoo biosensor maintain contact with the skin by means of physical adhesion (van der Waals forces), without the use of glue, gel, or other solutions. In embodiments, the tattoo sensors may be surrounded by, and their patterned leads may be covered in, a biocompatible adhesive layer.

Further disclosed is a electrophysiological monitoring system including the tattoo sensor device which is capable of monitoring and processing ECG, EMG, or EEG signals, and wireless communication with a monitor network. The system includes a reusable monitor configured for direct electrical connection with a contact region of the tattoo sensor device (i.e. electrical connection between the monitor and the contact region of the tattoo sensor does not utilize wires or leads). The system is suitable for long-time monitoring of a patient's vital parameters in ambulatory, in-home, or outpatient settings.

The embodiments disclosed herein may be summarized as follows.

### Embodiment 1

A device (100) for sensing electrophysiological signals, the device cooperating with a signal monitor (200), the device comprising:

a substrate sheet (104) having a backing sheet (104a) and a releasable coating layer (104c) formed on the backing sheet;

a conductive polymer pattern (102) formed on the releasable coating layer, the pattern including a plurality of sensor regions (110) each connected to a patterned lead (120) having a terminus (130) adjacent to a common contact region (140);

a plurality of sensor contacts (150) arranged within the contact region, each terminus of the patterned leads in electrical communication with one of the sensor contacts; and,

wherein the sensor contacts are configured for direct electrical connection to the signal monitor.

### Embodiment 2

The device of Embodiment 1, wherein the conductive polymer pattern and the releasable coating layer have a combined thickness of less than 1 micrometer.

### Embodiment 3

The device of Embodiment 1 or 2, wherein the plurality of sensor regions consists of two, three, four, five, or six sensor regions.

### Embodiment 4

The device of any one of Embodiments 1 to 3, wherein the conductive polymer is poly(3,4-ethylenedioxythiophene):poly(styrenesulfonate).

### Embodiment 5

The device of any one of Embodiments 1 to 4, wherein the contact region comprises a nonconductive support layer (160) to which the sensor contacts are connected.

### Embodiment 6

The device of any one of Embodiments 1 to 5, wherein the sensor contacts are configured for snap-fitting to the signal monitor.

### Embodiment 7

A wireless electrophysiological monitoring system (500), comprising:

a conductive polymer pattern (102) configured for transfer to the skin of a patient, the pattern including a plurality of sensor regions (110) each connected to a patterned lead (120) having a terminus (130) adjacent to a common contact region (140);

a plurality of sensor contacts (150) arranged within the contact region, each terminus of the patterned leads in electrical communication with one of the sensor contacts;

an electrophysiological monitor (200) having a plurality of monitor contacts (230) and configured to adhere to the skin of the patient such that one of the monitor contacts is in electrical connection with each of the sensor contacts, the monitor further including an integrated circuit (240) configured to digitize at least one of ECG, EEG, or EMG signals, a memory (260), and a transceiver (270) configured for wireless transmission of the digitized signals; and,

a mobile communication device (300) having a transceiver (370) configured for wireless communication with the monitor and a processor (310) configured to process the digitized signals, the mobile communication device configured to transmit the processed signals to a monitor network (400).

### Embodiment 8

The wireless electrophysiological monitoring system of Embodiment 7, wherein the plurality of sensor regions consists of two, three, four, five, or six sensor regions.

### Embodiment 9

The wireless electrophysiological monitoring system of Embodiment 7 or 8, wherein the monitor includes one or more integrated circuits (240) and is configured to digitize ECG, EEG, and EMG signals.

### Embodiment 10

The wireless electrophysiological monitoring system of any one of Embodiments 7 to 9, wherein the monitor includes a motion sensor (250).

### Embodiment 11

The wireless electrophysiological monitoring system of any one of Embodiments 7 to 10, wherein the mobile communication device is configured to continuously transmit the processed signals to the monitor network.

### Embodiment 12

The wireless electrophysiological monitoring system of any one of Embodiments 7 to 11, wherein the transmission of signals to the monitor network is accompanied by an indicator of at least one of: cardiac arrhythmia, ECG shape abnormality, respiration rate, heart rate, blood pressure, physical activity index, detected fall, or pre-seizure condition.

Further disclosed are methods of monitoring electrophysiological signals using the system of any one of Embodiments 7 to 12.

The following publications are hereby incorporated herein by reference in their entirety. In the case of any conflict between this document and the disclosure of the below references, this document controls.
Ameri et al. (2017), “Graphene Electronic Tattoo Sensors” (ACS Nano July 2017).
Bareket et al. (2016), “Temporary-tattoo for long-term high fidelity biopotential recordings” (Nature Scientific Reports, 2016, 6:25727, DOI: 10.1038/srep25727).
Casson et al. (2016), “Five day attachment ECG electrodes for longitudinal bio-sensing using conformal tattoo substrates” (IEEE Sensors Journal, DOI 10.1109/JSEN.2017.2650564).
Greco et al. (2011), “Ultra-thin conductive free-standing PEDOT/PSS nanofilms” (Soft Matter, 2011, 7, 10642).
Zucca et al. (2015), “Conformable Electronics: Tattoo Conductive Polymer Nanosheets for Skin-Contact Applications” (Adv. Healthcare Mater. July 2015, 4: 983).

### BRIEF DESCRIPTION OF THE DRAWINGS

FIG. 1 is a top plan view of a device for sensing electrophysiological signals.

FIG. 2 is an enlarged cross-sectional view along the line II-II of FIG. 1.

FIG. 3A shows the device being worn by a patient; and FIG. 3B shows a portion of an electrophysiological monitoring system in use.

FIG. 4 is a top plan view of another embodiment of the device.

FIG. 5 is a top plan view of another embodiment of the device.

FIG. 6 is a schematic representation of the electrophysiological monitoring system.

FIGS. 7A-7D are front, rear, side, and perspective views, respectively, of a monitor of the system.

FIGS. 8A-8L are screen displays of a mobile software application of the system.

FIGS. 9A-9F are screen displays presented to clients of a monitor network of the system.

### LIST OF DRAWING REFERENCE NUMERALS

100 device
102 conductive polymer pattern
104 substrate sheet
   104a backing sheet
   104b water-soluble layer
   104c releasable coating layer
106 adhesive layer
110 sensor region
120 patterned lead
130 terminus
140 contact region
150 sensor contact
160 nonconductive support layer
200 electrophysiological monitor
210 rear face
220 adhesive
230 monitor contact
240 integrated circuit
250 motion sensor
260 memory
270 transceiver
300 mobile communication device
310 processor
320 display
370 transceiver
380 battery
400 monitor network
410 cloud
420 user client
430 medical professional client
440 other client
500 electrophysiological monitoring system

### DETAILED DESCRIPTION OF THE INVENTION

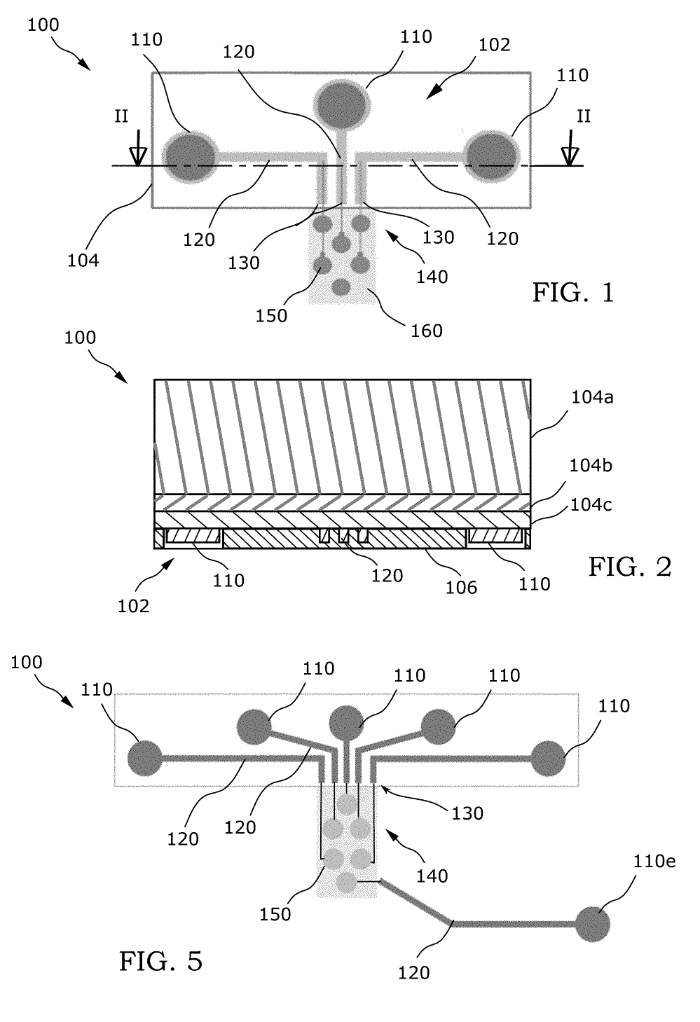

Referring initially to FIGS. 1-2, there are illustrated top plan and enlarged cross-sectional views, respectively, of a device for sensing electrophysiological signals (biosignals), the device generally designated as 100. Device 100 comprises a conductive polymer pattern 102 on a substrate sheet 104, such as decal transfer paper which is commonly used for temporary tattoos. Conductive polymer pattern 102 includes a plurality of sensor regions 110 (hereinafter referred to as sensors), three sensors 110 being present in the shown embodiment. In other embodiments, 2, 4, 5, 6, or another number of sensors 110 may be included.

In embodiments, conductive polymer pattern 102 comprises a high-conductivity polymer complex poly(3,4-ethylenedioxythiophene):poly(styrenesulfonate) (PEDOT:PSS). The polymer pattern may be ink-jet patterned onto the decal transfer paper substrate sheet 104, in one of the manners described by Zucca, et al. (2015). Substrate sheet 104 may comprise three layers: i) a backing sheet 104a, such as a water-permeable paper; ii) a water-soluble layer 104b, such as a starch-dextrin coating, and iii) a releasable coating layer 104c, such as ethylcellulose (EC). Polymer pattern 102 is patterned on releasable coating layer 104c of substrate sheet 104.

In further embodiments, an adhesive layer 106 is applied to device 100 after the polymer pattern has been patterned on substrate 104. Adhesive layer 106 may be a double-sided biocompatible adhesive one side of which is adhered to layer 104c and pattern 102 and the other side of which is configured to adhere to the skin of the patient. Preferably, before application of adhesive layer 106 to device 100, holes are cut in adhesive layer 106 corresponding to the locations of sensors 110 of pattern 102, thereby permitting direct contact between sensors 110 and the skin when in use. Adhesive layer 106 provides longer term durable adhesion of device 110 and protects small features of the pattern, such as patterned leads 120, from wear.

FIG. 2 shows a cross-sectional view along line II-II of FIG. 1, the view enlarged in height to better illustrate the thickness of the layers of the device (contact region 140 not shown for clarity). In the orientation shown, the bottommost layer is in contact with the skin of the patient during application and use. In embodiments, conductive polymer pattern 102 and the releasable coating layer 104c have a combined thickness of less than 1 micrometer, less than 750 nanometers (nm), less than 700 nm, less than 650 nm, or less than 600 nm. In an example embodiment, conductive polymer pattern 102 has a thickness of about 250 nm and releasable coating layer 104c has a thickness of about 360 nm, giving the transferred tattoo a total thickness of about 610 nm. In other embodiments, adhesive layer 106 also has a thickness of less than 1 micrometer.

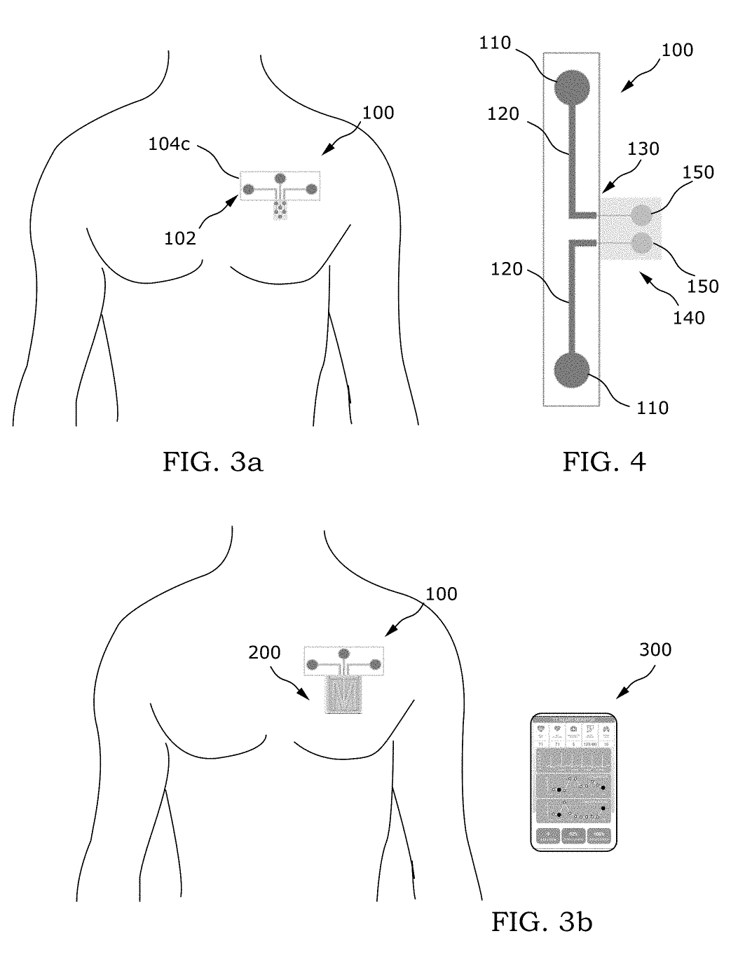

FIG. 3A shows conductive polymer pattern 102 being worn by a patient, in a manner suitable for electrocardiography (ECG or EKG). Conductive polymer pattern 102 is transferred to the skin of a patient in the manner of applying a temporary tattoo. Device 100 is put in contact with the skin of a patient such that the backing sheet 104a (not shown, see FIG. 2) is away from the body. When backing sheet 104a is moistened with water, the water-soluble layer 104b dissolves and releasable coating layer 104c and conductive polymer pattern 102 are released from substrate sheet 104 and transferred to the patient's skin. Backing sheet 104a may be moistened, such as with a wet cloth, and pressure applied for approximately 30 seconds, by which time the polymer pattern 102 and releasable coating layer 104c will be transferred to the patient. Backing sheet layer 104a may then be removed and discarded.

FIG. 3B shows the patient wearing device 100 with a cooperating monitor 200 and mobile communication device (MCD) 300, which are further discussed below.

Referring again to FIG. 1, each sensor 110 of the pattern is connected to a patterned lead 120 formed of the same conductive polymer as sensor 110. Each patterned lead 120 has a terminus 130 adjacent to or within a common contact region 140. In an embodiment each sensor 110 has a diameter of about 15 mm. In the embodiment of FIG. 1 the spacing between sensors 110 on the far left and far right is about 80 mm. In the shown embodiment, patterned leads 120 have a width on the order of 2.5 mm. In other embodiments, patterned leads may be wider to support a longer length of lead.

Each terminus 130 is in electrical connection with a sensor contact 150 located within contact region 140 (six of sensor contact 150 are present in the shown embodiment). In an embodiment, ultrathin wires connect each terminus 130 to a sensor contact 150. In another embodiment, the wires are sandwiched between two layers of tape which provide support and protection for the wires. In embodiments, one end of each wire is located between a terminus 130 of conductive polymer pattern 102 and releasable coating layer 104c. The other end of each wire is connected to a sensor contact 150, such as by clipping between a male and female component of sensor contact 150.

In another embodiment of the electrical connection between terminus 130 and sensor contact 150, the connection may include a substantially planar layer of conductive polymer, such as PEDOT:PSS, rather than ultrathin wires. In embodiments, the conductive polymer may be printed on a support layer to which sensor contacts 150 are connected.

Contact region 140 is configured such that a monitor having electrical contacts for direct electrical connection with some or all of sensor contacts 150 may be easily and conveniently positioned over contact region 140 (see FIG. 3B). This direct electrical connection is made without the use of wires or leads. In embodiments, sensor contacts 150 are configured for snap-fitting to the signal monitor, for example sensor contacts 150 may be a male or female electroconductive stud configured for snap-fitting to the complementary female or male monitor contact 230. Each sensor contact 150 may be sized on the order of 3 mm diameter, and arranged with an interstitial spacing of around 5-10 mm.

In an embodiment, contact region 140 comprises a nonconductive support layer 160 to which the sensor contacts are connected. For example support layer 160 may be made from polyethylene terephthalate (PET), and sensor contacts 150 may be electroconductive studs having front and back components which are connected with the PET support layer between the front and back components. In an embodiment, contact region 140 may not be attached to the skin of the patient and may be supported by connection to the tattoo substrate 104c.

FIGS. 4 & 5 are top plan views of additional embodiments of device 100, the conductive polymer pattern 102 of FIG. 4 having two sensors 110 and that of FIG. 5 having six sensors 110. The device of FIG. 4 may be transferred to a patient's skin near a skeletal muscle (such as locations on an arm or leg) for use in electromyography (EMG). In the shown embodiment, sensors 110 have a spacing of about 12 cm and are configured for placement on a biceps muscle. In another example embodiment, sensors 110 have a spacing of about 20 cm for placement on a quadriceps muscle. The device of FIG. 5 may be transferred to a patient's scalp for use in electroencephalography (EEG). Sensor 110e may be transferred to the patient's earlobe, and used as a reference electrode. Sensors 110 may be otherwise arranged for detection of biosignals, for example any number from three to six of sensors 110 may be arranged in accordance with the 10-20 electrode placement system for EEG. In other embodiments, the size, shape, number, and arrangement of sensors 110, as well as that of contacts 150 may be custom designed for specific applications.

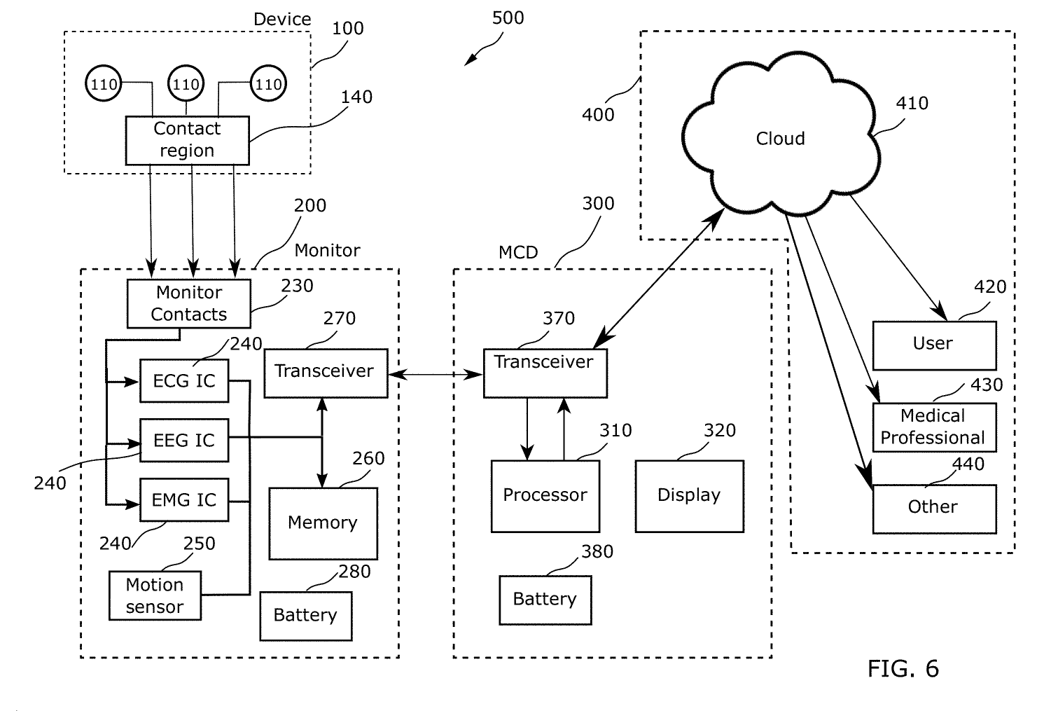

FIG. 6 is a schematic representation of an electrophysiological monitoring system 500 including a device 100 generally as described above.

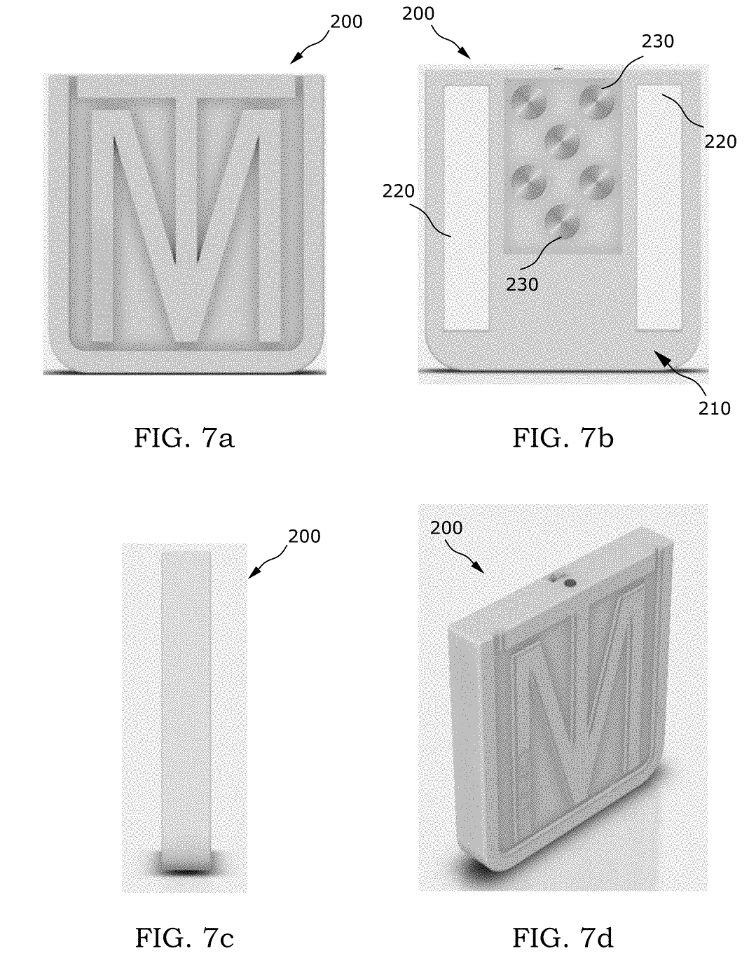

An electrophysiological monitor 200 is configured to adhere to the skin of the patient and connect electrically with conductive polymer pattern 102, via contact region 140. FIGS. 7A-7D are front, rear, side, and perspective views, respectively, of monitor 200. A plurality of electrical monitor contacts 230 are located on the rear face 210 of the monitor (six contacts 230 shown in FIG. 7B). Contacts 230 are located so that when monitor 200 is positioned over contact region 140 and adhered to the patient's skin with rear face 210 facing the skin, one of monitor contacts 230 is in electrical connection with each sensor contact 150 which is in electrical connection with one of the patterned leads 120 of the conductive polymer pattern 102. For example, monitor 200 may have six monitor contacts 230. When used with a tattoo sensor having six of sensor 110, each monitor contact is in electrical connection with one sensor contact 150. When the same example monitor is used with a tattoo sensor having two or three of sensor 110, one monitor contact 230 is in electrical connection with each electrically connected sensor contact 150, while the remaining four or three monitor contacts 230 are unused (not connected).

Monitor contacts 230 may be electroconductive studs having an overall diameter of about 3.5 mm. Strips of biocompatible adhesive 220 on rear face 210 of monitor 200 enable ready attachment to the skin of the patient. In embodiments, adhesive 220 is readily replaceable each time monitor 200 is removed from the patient's skin, or as otherwise desired to maintain proper adhesion. Monitor 200 and/or contact region 140 may have indicia marking proper placement of monitor 200 over contact region 140. In an embodiment, monitor 200 has a height of 45 mm, a width of 40 mm, and a thickness of 7 mm.

Referring again to FIG. 6, electrophysiological monitor 200 includes at least one integrated circuit (IC) 240 configured to digitize biosignals received from sensors 110 which may be any one of ECG, EEG, or EMG signals. In the shown embodiment, monitor 200 includes three of IC 240, each of which digitizes one of ECG, EEG, or EMG signals. In another embodiment, a single IC 240 may be configured to digitize all of ECG, EEG, and EMG signals. Other IC configurations maybe readily envisioned to achieve the same result. IC 240 may further perform additional functions, such as signal amplification, filtering, lead-off detection, signal resampling, impedance measurement, etc. Signals processed by IC 240 in any of the above-mentioned manners are referred to herein as digitized signals.

In an embodiment, monitor 200 includes a motion sensor 250 such as an accelerometer or piezoelectric sensor, for detecting sudden movements of the patient, such as a fall.

Monitor 200 is powered by a replaceable or rechargeable battery 280, such as a standard button cell battery. Multiple batteries 280 may be provided with monitor 200 so that while a first battery is installed in monitor 200 a second battery may be recharged and ready to replace the first (in use) battery as needed. In this manner, monitor 200 may be used substantially uninterrupted for prolonged periods of time (up to several years).

A transceiver 270 in monitor 200 wirelessly transmits digitized signals or motion sensor data to a mobile communications device (MCD) 300, such as a cellular telephone, tablet, or the like. In one embodiment, transceiver 270 uses the Bluetooth Low Energy (BLE) specification to prolong battery life of the monitor. In embodiments, a BLE transceiver may be left on continuously or may alternate between a full power “wake” mode and a lower power “sleep” mode. In other embodiments, transceiver 270 may use wireless internet communication, standard Bluetooth, or other communication protocols known in the art.

Monitor 200 includes a memory 260, such as a flash memory, ROM, EEPROM, or the like. In one embodiment, memory 260 is an SD card, and digitized signals may be stored internally to monitor 200 for up to a 24 hour period.

In an embodiment, monitor 200 has two operating modes, Holter mode and monitor mode. When operated in Holter mode, digitized signals are stored on memory 260 of monitor 200 for a period of time such as 12, 24, 36, or 48 hours. When in Holter mode monitor 200 stores sensor data without transferring data to MCD 300 or other devices or networks. When monitor 200 is operated in monitor mode, digitized signals may be temporarily stored on memory 260 of monitor 200 and are transferred to MCD 300 either in pseudo-real time or as soon as a network connection is available.

MCD 300 includes a transceiver 370 for wirelessly receiving and transmitting signals to or from monitor 200 and a monitor network 400, which may be a cloud network or other internet network. While transceiver 370 is referred to herein in the singular, transceiver 370 may comprise multiple distinct hardware elements for communication via various protocols. For example, transceiver 370 may include a BLE transceiver for sending/receiving signals to/from monitor 200; a wireless network interface which supports a typical wireless local area network (WLAN), for example, Wi-Fi, or some other wireless local network capability, like, for example, femtocell or picocell wireless, Wireless USB, etc. for transmitting signals to monitor network 400; and/or an interface to a cellular network for transmitting signals to the monitor network.

In addition to transceiver 370 receiving digitized signals from monitor 200, transceiver 370 may transmit signals or instructions to monitor 200, such as to change the operational configuration of IC 240 (e.g., changing gain, sampling rate, or filter settings), to alternate between Holter and monitor modes, to query status of connections or battery levels, to request data transfer from memory 260, or other operational instructions.

MCD 300 further includes a processor 310 configured to process the digitized signals received from monitor 200 by transceiver 370. Processing performed by processor 310 may include signal filtering; artifact removal; comparison of digitized signals with databases of normal and pathologic ECG/EMM/EEG signals; template matching; detecting ECG abnormalities, such as arrhythmias and abnormalities in the morphology (“shape”) of the ECG wave which may be predictive of critical cardiac events; determining vital signs such as heart rate, respiration rate, physical activity index or blood pressure; detecting falls; and applying fast Fourier transform (FFT) to extract amplitudes or relevant frequencies for EMG and EEG signals. Outputs of any of the aforementioned processing performed on the MCD are referred to hereinafter as processed data.

MCD 300 further includes a display 320, which may display to the user certain digitized signals or processed data, and a battery 380. It is particularly advantageous to perform signal processing on processor 310 of MCD 300 rather than on monitor 200 itself, due to the high speed and processing power of commercially available MCDs at relatively low cost as compared to processors customized to specific applications. By minimizing the signal processing performed on-board monitor 200, the time before discharge of battery 280 may be extended and the overall size of the monitor reduced. Signal processing may be controlled via a mobile software application (app), suitable for installation on commercially available MCDs. In an embodiment, the MCD is dedicated for use with system 500.

Signals processed by processor 310 are transmitted by transceiver 370 to monitor network 400. In addition, unprocessed digitized signals received by MCD 300 may be transmitted to monitor network 400 for analysis or processing outside of MCD 300, such as by a medical professional connected to monitor network 400. When monitor 200 is in monitor mode and wireless communication channels are active, data transfer from MCD 300 to monitor network 400 is continuous.

In embodiments, monitor network 400 includes a cloud 410 which may be accessed by a number of clients such as a user 420 (the patient wearing device 100); a medical professional 430, which may be an individual or team of doctors, a hospital network, out-patient care provider, or similar; and other clients 440 such as an emergency points of contact, lay caregivers, patient supervisors, etc.

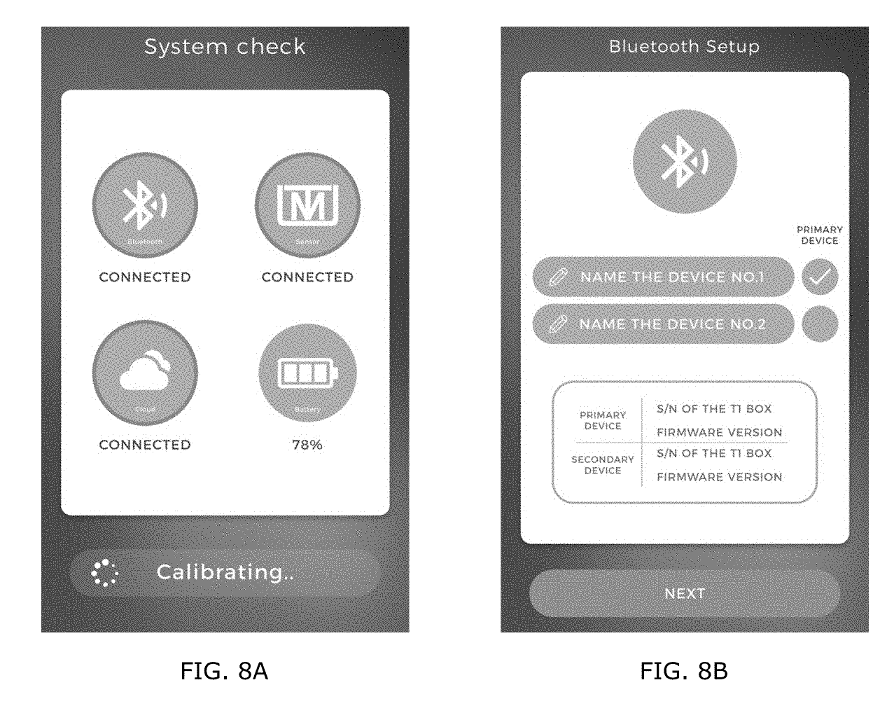

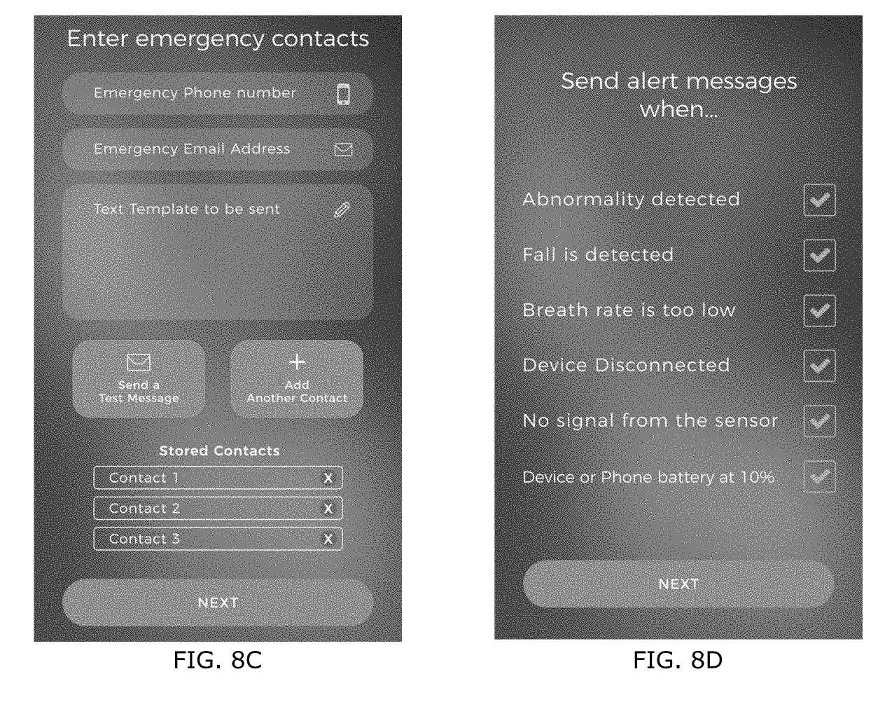

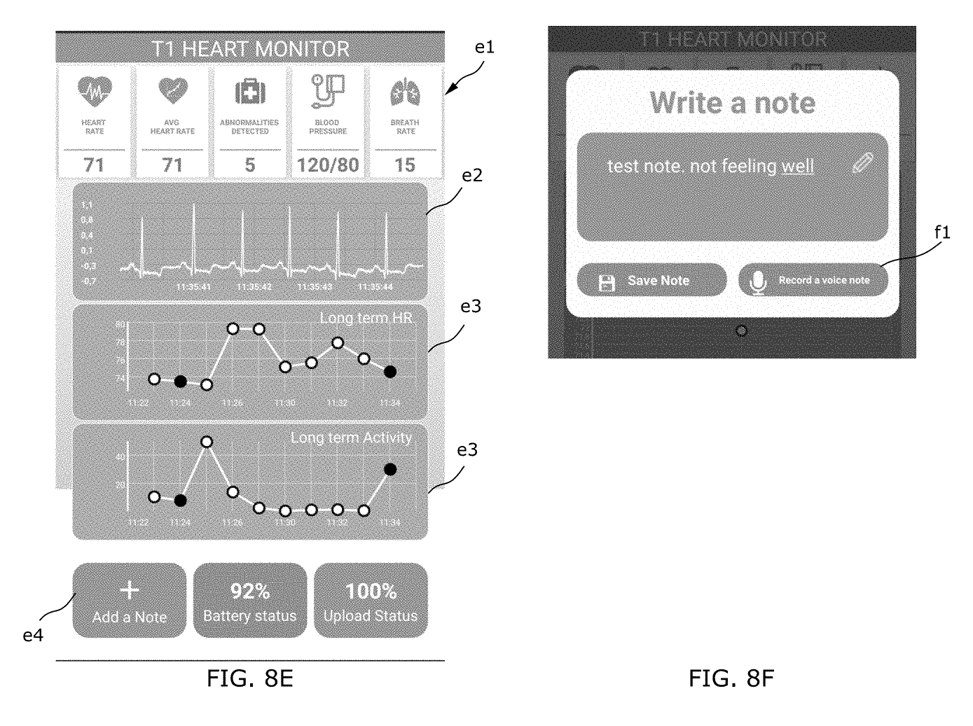

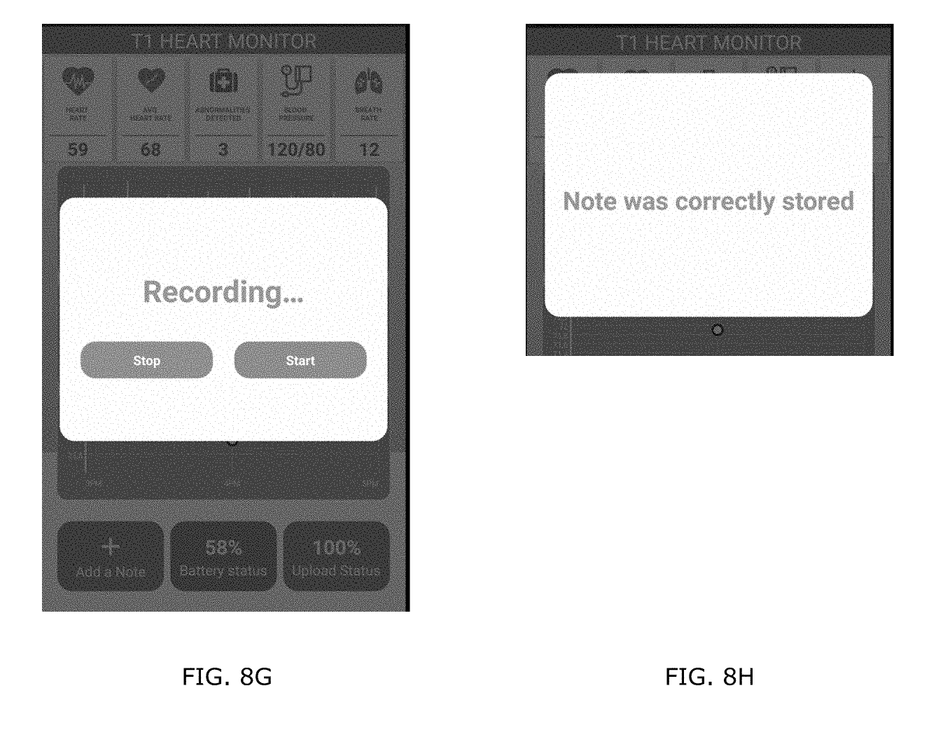

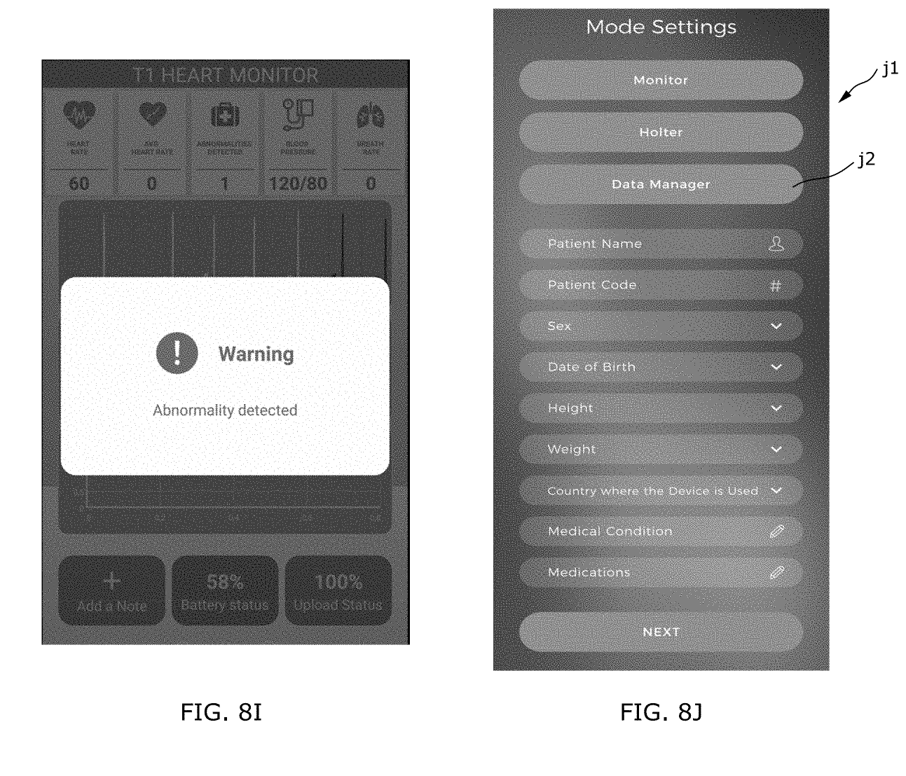

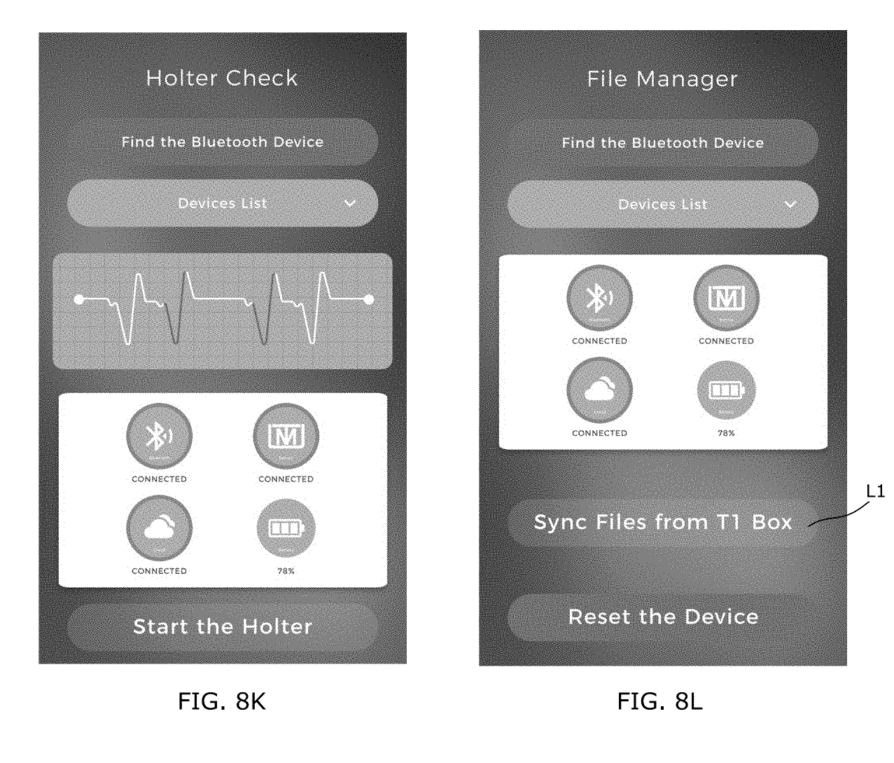

FIGS. 8A-8I are example screen displays of a mobile software application (app) shown on display 320 (see FIGS. 3B & 6) of MCD 300 when monitoring system 500 is in use. FIG. 8A shows a screen displaying various system status indicators, such as connection to Bluetooth or other short-distance wireless communication link, connection to the monitor, connection to the monitor network, and battery charge level. FIG. 8B shows a screen for configuring wireless connection to one or more monitors. FIG. 8C shows a screen for configuring emergency contacts to whom emails or SMS (text) alert messages may be sent notifying them that intervention may be required. FIG. 8D shows a configuration screen for selecting when to send alert messages to the emergency contact. Alerts may be sent for detected conditions such as a ECG/EMG/EEG signal abnormality, detected fall, low breathing rate, disconnected monitor, sensor signal not detected, low battery on monitor or MCD, or other conditions of interest not shown.

FIG. 8E is a default monitor display showing a plurality of processed data indicators (e1), such as heart rate, blood pressure, breathing rate, and number of abnormalities detected. The display also shows several graphs over time, such as detected signal from sensors (e2) and results of data processing (e3, long term heart rate and physical activity in the shown display). Occurrences of abnormalities may indicated on the graphs such as in a contrasting color or with distinct markers, as shown in e3. The status indicators and graphs to be displayed on the default screen may be selected by the user. Button e4 at the bottom of the screen allows the user to add a note, and calls the display of FIG. 8F. The user may type a text note, or record a voice note (button f1, which calls the display of FIG. 8G). Text or voice notes may be stored locally or transmitted to the monitor network. A confirmation of a note being stored is shown on the screen of FIG. 8H.

If an abnormality is detected, a warning message is displayed on the MCD, such as that shown in FIG. 8I. The MCD may simultaneously provide other indications to the user, such as a vibration or an audible alarm. In addition to the warning, emergency contacts are notified when abnormalities occur, as configured by the user on the screens of FIGS. 8C-8D. When a text message is sent to an emergency contact, the screen may display a notification that the message was sent. When monitor 200 is in Holter mode, alarms are not displayed to the user and emergency contact notifications are not sent.

FIG. 8J shows a display which enables the MCD user to configure the monitor to operate in Holter or monitor modes (buttons j1). When operating in Holter mode, the user may check monitor status and start or stop Holter mode using the screen shown in FIG. 8K. The user may initiate transfer of data stored on memory 260 during a Holter mode session using button j2 (FIG. 8J) or the file manager screen shown in FIG. 8L (button L1).

An interface module enables client devices (computers, servers, mobile phones and the like) to manage connection to and view data received from the monitor network. The module may be installed locally on a client device or may be remotely hosted and accessed by the client device via an internet browser. The module manages communication between devices within the monitor network, synchronizes data between the MCD and client devices, and provides clients with a graphical user interface (‘dashboard’) which may be customized for the type of client (user of device, medical professional, emergency contact, etc.). The module enables multiple clients to access session data either in real-time or asynchronously.

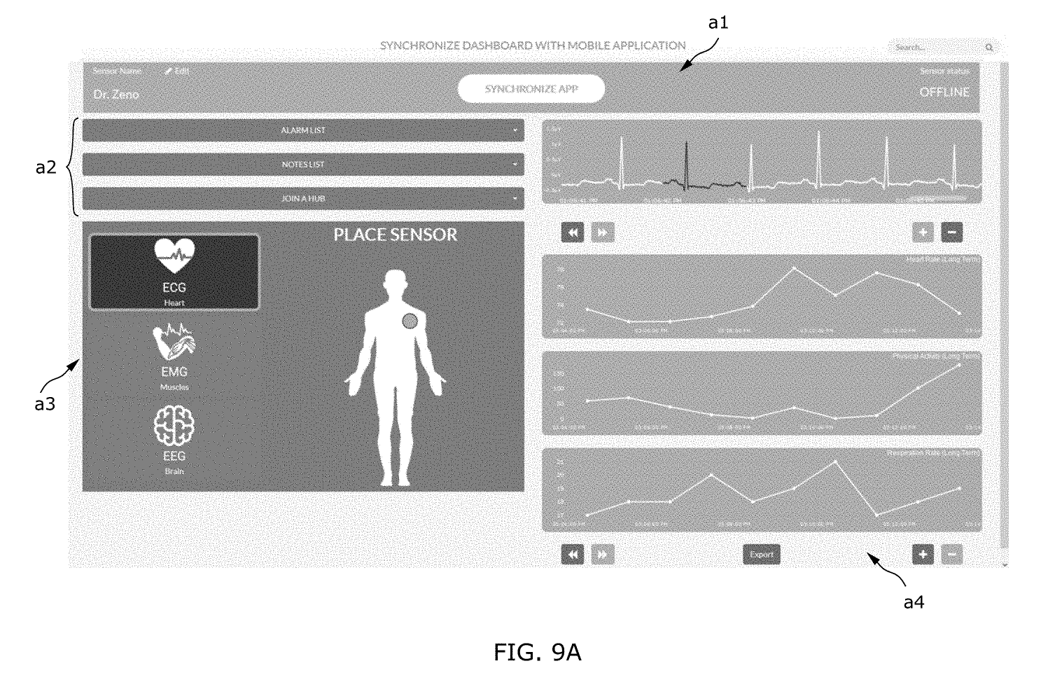

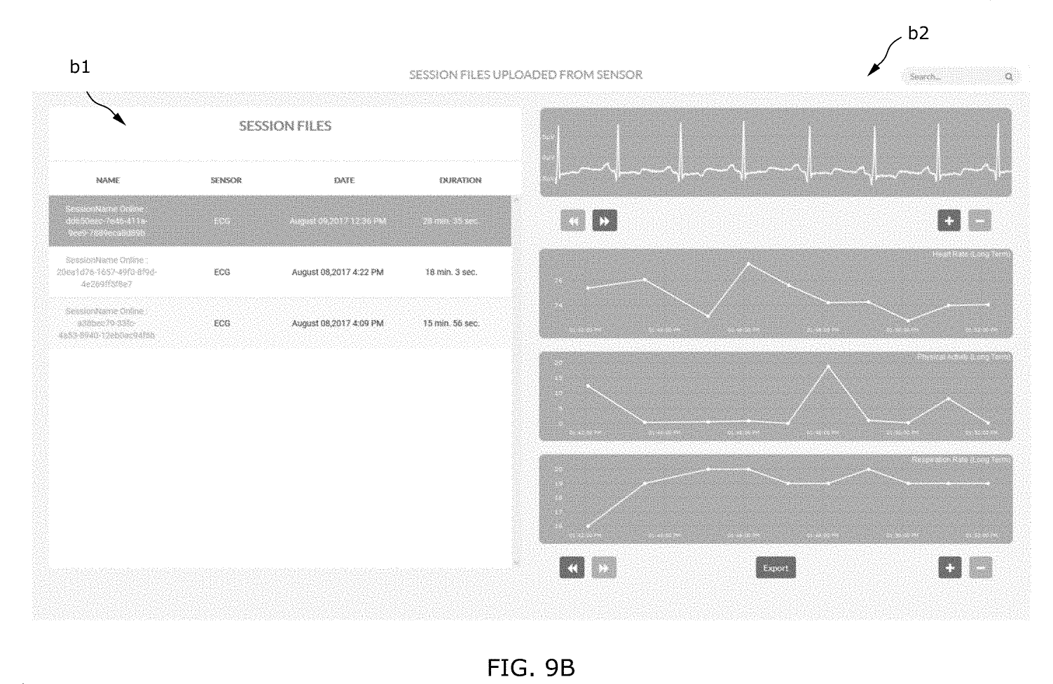

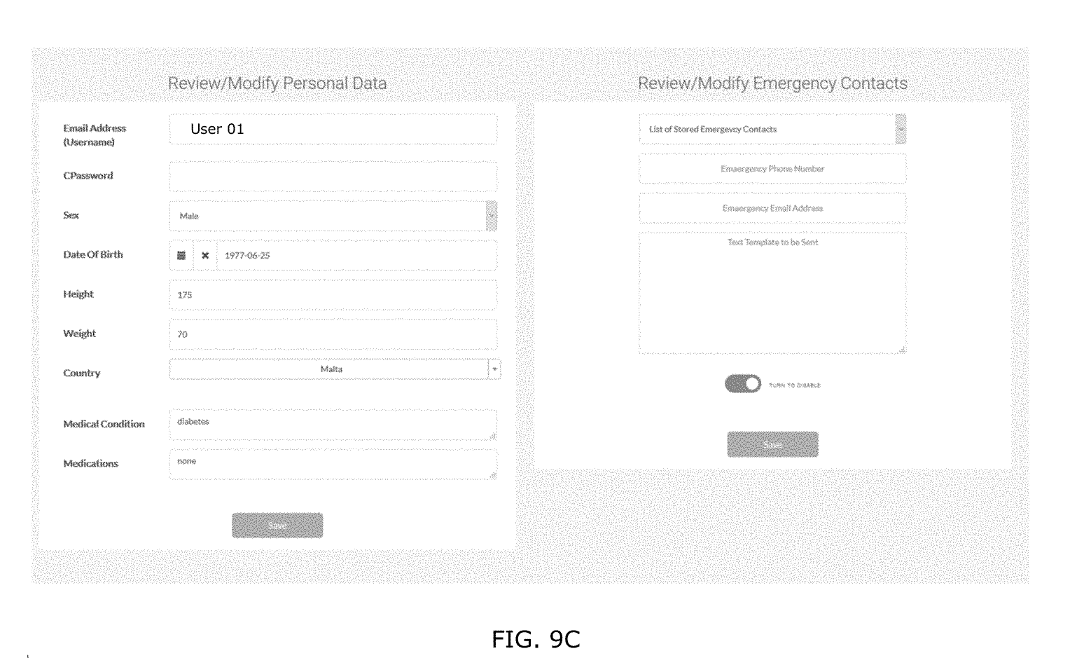

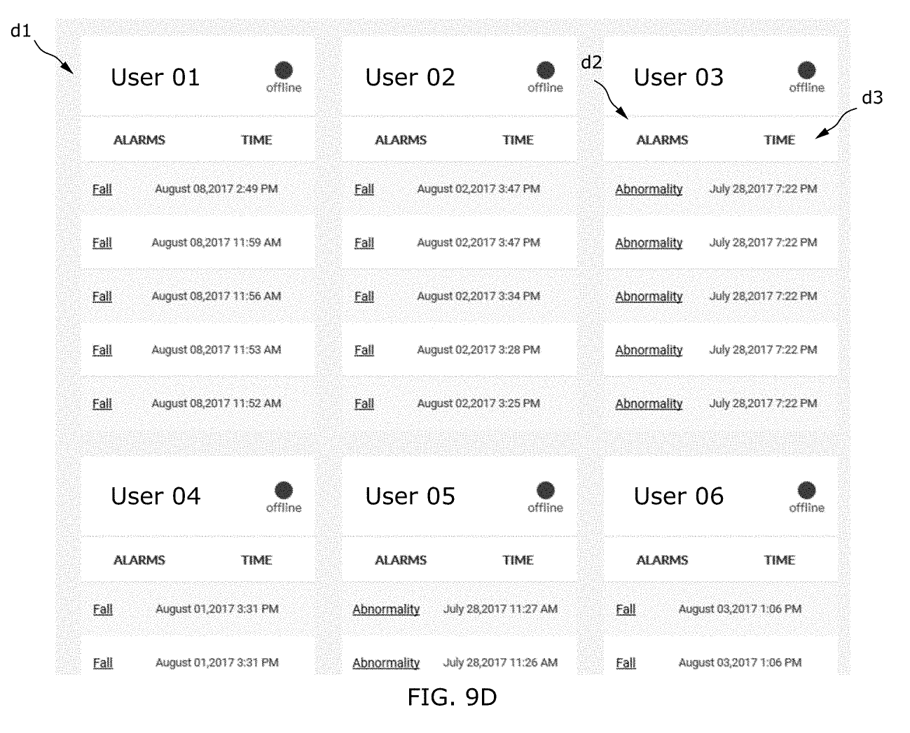

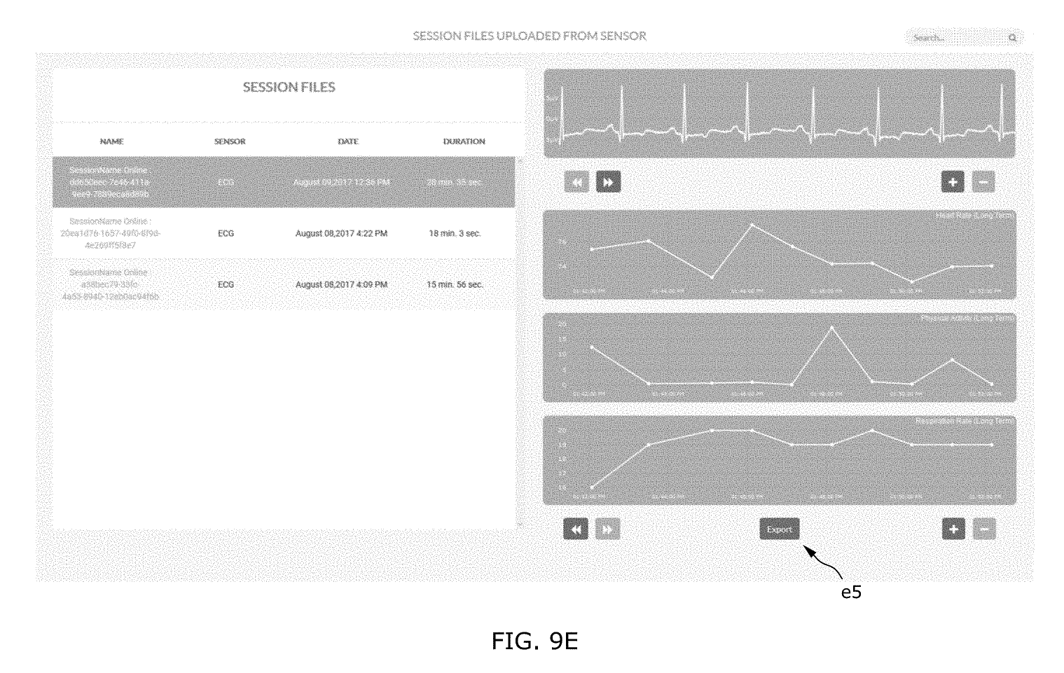

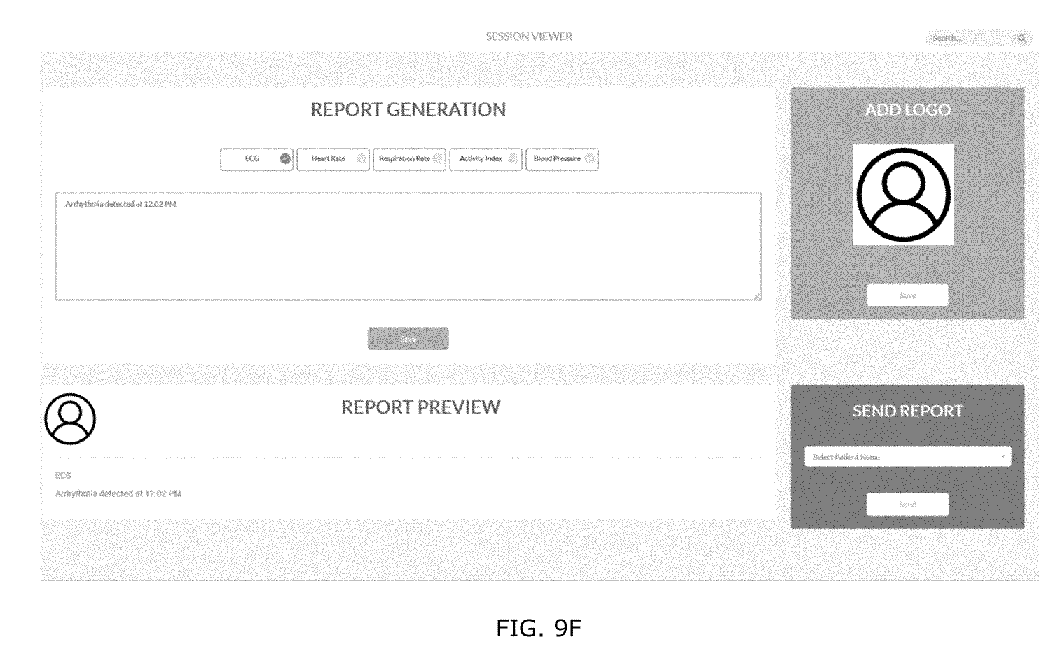

FIGS. 9A-9F are example dashboards presented to clients 420, 430, 440 when connected to monitor network 400 (see FIG. 6). FIG. 9A is a default dashboard shown to a user 420. A button a1 allows the user to synchronize their dashboard display with the app of MCD 300, ensuring display of the most up to date data and user settings. Pull down menus or buttons a2 provide additional functionality such as allowing the user to display lists of alarms or notes, or request to join a specific hub or group of network users (e.g. group of patients in the same hospital). The type or location of sensor being used is selected in region a3. Region a4 displays time logs of sensor data and processed data, which may be scrolled and zoomed to display different time windows using the arrow and +/− buttons. Session data may also be exported for saving locally.

FIG. 9B shows a dashboard from which user 420 may select from a list of sessions (b1) to be displayed. Sensor data and processed data from the selected session is displayed in region b2 in a similar manner as described for region a4 of FIG. 9A.

FIG. 9C shows a dashboard from which user 420 may review or edit personal data and emergency contact information. In a preferred embodiment, edits made by the user in their dashboard are automatically synchronized with the MCD app.

FIG. 9D shows a partial default dashboard shown to a medical professional 430, displaying all users 420 in the monitor network associated with professional 430, and a user summary box d1 for each associated user. Each user summary box gives lists of alarms (d2) and times of alarm events (d3) for the associated user. Alarms may be falls, abnormal events, or other conditions as discussed above. Professional 430 may navigate to an alarm viewer or a session viewer for an individual user 420, shown in FIG. 9E, which displays time logs associated with the selected alarm or session, similar to those displayable by the user (FIG. 9B).

In an embodiment, the time logs associated with an alarm include 10 minutes of processed data, such as 5 minutes before and 5 minutes after the alarm event. In embodiments, time logs associated with sessions include all processed data from a recording session, such as periods of time while the user is awake, in between battery changes, the duration of a Holter mode session, a session recorded during a particular physical activity, a periodic monitoring session or similar. Recording sessions are typically of longer duration than alarm sessions, and may for example be 1, 8, 10, 12 or 24 hours in length.

Medical professionals may export alarm or session data (button e5) for saving locally, such as for further processing.

FIG. 9F shows a dashboard from which user 420 may generate a report related to a particular session or alarm. Reports may contain analysis of data, related medical notes, or the like. The report may be saved and sent to other clients 420, 430, 440.

A dashboard for another type of client 440, may provide limited access to session or alarm information viewable by the user or medical professional, or may be configurable by the user to set access permissions for different types of client.

In terms of use, a method of monitoring electrophysiological signals includes: (refer to FIGS. 1-9)
a. providing an electrophysiological monitoring system of any one of Embodiments 8-13;
b. transferring at least a portion of the conductive polymer pattern (102) to the skin of a patient;
c. adhering the electrophysiological monitor (200) to the skin of the patient such that one of the monitor contacts (230) is in electrical connection with each of the sensor contacts (150);
d. digitizing, by the integrated circuit (240) of the monitor, at least one of ECG, EEG, or EMG signals sensed by at least one of the sensor regions (110) of the conductive polymer pattern;
e. wirelessly transmitting, by the transceiver (270) of the monitor, the digitized signals to the mobile communication device (300);
f. processing the digitized signals with the processor (310) of the mobile communication device; and,
g. transmitting, by the mobile communication device, the processed signals to the monitor network (400).

The method further including,
in (g), continuously transmitting the processed signals to the monitor network.

The method further including,
transmitting to the monitor network, by the mobile communication device, an indicator of at least one of: cardiac arrhythmia, ECG shape abnormality, respiration rate, heart rate, blood pressure, physical activity index, or detected fall.

The method further including,
after (g), further processing transmitted processed signals by a module in communication with the monitor network.

As used in this application, the term “about” or “approximately” refers to a range of values within plus or minus 10% of the specified number. As used in this application, the term “substantially” means that the actual value is within about 10% of the actual desired value of any variable, element or limit set forth herein.

The embodiments of the device, system, and method of use described herein are exemplary and numerous modifications, combinations, variations, and rearrangements can be readily envisioned to achieve an equivalent result, all of which are intended to be embraced within the scope of the appended claims. Further, nothing in the above-provided discussions of the device, system, and method should be construed as limiting the invention to a particular embodiment or combination of embodiments. The scope of the invention is defined by the appended claims.

1. A device for sensing electrophysiological signals, the device cooperating with a signal monitor, the device comprising:
a substrate sheet having a backing sheet and a releasable coating layer formed on the backing sheet;
a conductive polymer pattern formed on the releasable coating layer, the conductive polymer pattern including a plurality of sensor regions each connected to a patterned lead having a terminus adjacent to a common contact region;
the common contact region located on a nonconductive support layer distinct from the releasable coating layer, a plurality of sensor contacts connected to the nonconductive support layer within the common contact region, each terminus of the patterned leads in electrical communication with one of the plurality of sensor contacts via a conductive lead distinct from the patterned leads, the conductive lead supported by the nonconductive support layer; and,
wherein the plurality of sensor contacts are configured for snap-fitting to the signal monitor.

2. The device of claim 1, wherein the conductive polymer pattern and the releasable coating layer have a combined thickness of less than 1 micrometer.

3. The device of claim 1, wherein the plurality of sensor regions consists of two, three, four, five, or six sensor regions.

4. The device of claim 1, wherein the conductive polymer pattern is formed of poly(3,4-ethylenedioxythiophene):poly(styrenesulfonate).

5. (canceled)

6. (canceled)

7. A wireless electrophysiological monitoring system, comprising:
a conductive polymer pattern configured for transfer to the skin of a patient, the conductive polymer pattern including a plurality of sensor regions each connected to a patterned lead having a terminus adjacent to a common contact region;
the common contact region located on a nonconductive support layer, a plurality of sensor contacts connected to the nonconductive support layer within the common contact region, each of the plurality of sensor contacts being a male or female electroconductive stud, each terminus of the patterned leads in electrical communication with one of the plurality of sensor contacts via a conductive lead distinct from the patterned leads, the conductive lead supported by the nonconductive support layer;
an electrophysiological monitor having a plurality of monitor contacts located on a rear face, each of the plurality of monitor contacts being a female or male electroconductive stud complementary to one of the plurality of sensor contacts, the electrophysiological monitor configured to be positioned over the common contact region such that the rear face faces the skin of the patient and one of the plurality of monitor contacts is in direct electrical connection with each of the plurality of sensor contacts, the electrophysiological monitor further including an integrated circuit configured to digitize at least one of ECG, EEG, or EMG signals, a memory, and a transceiver configured for wireless transmission of the digitized signals; and,
a mobile communication device having a transceiver configured for wireless communication with the electrophysiological monitor and a processor configured to process the digitized signals, the mobile communication device configured to transmit the processed signals to a monitor network.

8. The wireless electrophysiological monitoring system of claim 7, wherein the plurality of sensor regions consists of two, three, four, five, or six sensor regions.

9. The wireless electrophysiological monitoring system of claim 7, wherein the electrophysiological monitor includes one or more integrated circuits and is configured to digitize ECG, EEG, and EMG signals.

10. The wireless electrophysiological monitoring system of claim 7, wherein the electrophysiological monitor includes a motion sensor.

11. The wireless electrophysiological monitoring system of claim 7, wherein the mobile communication device is configured to continuously transmit the processed signals to the monitor network.

12. The wireless electrophysiological monitoring system of claim 7, wherein the transmission of signals to the monitor network is accompanied by an indicator of at least one of: cardiac arrhythmia, ECG shape abnormality, respiration rate, heart rate, blood pressure, physical activity index, detected fall, or pre-seizure condition.

13. A method of monitoring electrophysiological signals, the method comprising:
a. providing an electrophysiological monitoring system of claim 7;
b. transferring at least a portion of the conductive polymer pattern to the skin of a patient;
c. adhering the electrophysiological monitor to the skin of the patient such that one of the monitor contacts is in electrical connection with each of the plurality of sensor contacts;
d. digitizing, by the integrated circuit of the electrophysiological monitor, at least one of ECG, EEG, or EMG signals sensed by at least one of the sensor regions of the conductive polymer pattern;
e. wirelessly transmitting, by the transceiver of the electrophysiological monitor, the digitized signals to the mobile communication device;
f. processing the digitized signals with the processor of the mobile communication device; and,
g. transmitting, by the mobile communication device, the processed signals to the monitor network.

14. The method of claim 13 further including,
in (g), continuously transmitting the processed signals to the monitor network.

15. The method of claim 13 further including,
transmitting to the monitor network, by the mobile communication device, an indicator of at least one of: cardiac arrhythmia, ECG shape abnormality, respiration rate, heart rate, blood pressure, physical activity index, detected fall, or pre-seizure condition.

16. The method of claim 13 further including,
after (g), further processing transmitted processed signals by a module in communication with the monitor network.

17. The device of claim 1, wherein each terminus of the patterned leads is in electrical communication with one of the plurality of sensor contacts via a layer of conductive polymer formed on the nonconductive support layer.

18. The device of claim 1, wherein each terminus of the patterned leads is in electrical communication with one of the plurality of sensor contacts via a wire supported on the nonconductive support layer.

19. The wireless electrophysiological monitoring system of claim 7, wherein each terminus of the patterned leads is in electrical communication with one of the plurality of sensor contacts via a layer of conductive polymer formed on the nonconductive support layer.

20. The wireless electrophysiological monitoring system of claim 7, wherein each terminus of the patterned leads is in electrical communication with one of the plurality of sensor contacts via a wire supported on the nonconductive support layer.
